(19) United States
(12) Patent Application Publication
(10) Pub. No.: US 2023/0190264 A1
(43) Pub. Date: Jun. 22, 2023

(54) DEVICE AND METHOD FOR CINCHING SUTURES AND A QUICK-RELEASE MECHANISM

(71) Applicant: JIVATMA LLC, Birmingham, MI (US)

(72) Inventors: Manisha Kiran MEHTA, Mumbai (IN); Nishaki MEHTA, Birmingham, MI (US)

(21) Appl. No.: 18/085,929

(22) Filed: Dec. 21, 2022

Related U.S. Application Data
(60) Provisional application No. 63/292,639, filed on Dec. 22, 2021.

(51) Int. Cl. A61B 17/04

(52) U.S. Cl. CPC A61B 17/0487; A61B 2017/00477

(57) ABSTRACT

A flat or rounded or ellipsoidal or cylindrical or smooth device or body with no sharp edges for cinch suture knotting to secure sutures/lines/wires/threads and the like. The body eliminates the need for fastening or deploying manual knots. The suture knotting device comprises male and female separable parts that secure a catheter or any device or tissue or tissue to the skin or any body tissue. The separable parts can be gently pulled or twisted apart to adjust or remove the suture, depending on the clinical condition to thereby avoid pinching or cutting of skin and trauma to tissue while removing the device. The suture knotting device helps to deploy, adjust and remove sutures without requiring any sharp objects or a suture removal kit. The device allows a wide range of dexterity and maneuvering for physicians or nurses with ease and less pressure.

### REFERENCE TO RELATED APPLICATIONS

This application claims the benefit of U.S. provisional application Ser. No. 63/292,639 filed Dec. 22, 2021, the disclosure of which is hereby incorporated in its entirety by reference herein.

### TECHNICAL FIELD

### Field of the Invention

The present disclosure generally relates to medical and surgical devices, specifically to a cinch suture knotting capsule and method for securing suture threads/wires/lines/catheters to a catheter or any device to the skin or body tissue and to eliminate the need for manual knots.

### Background of the Invention

When an opening is created in tissue either through an intentional incision or an accidental wound or laceration, biological healing of the opening is commenced through the proximity of living tissue. If the opening is very large or if its location subjects the wound to continual movement, a physician will seek to approximate the sides of the incision or wound nearby to promote the healing process. The most prevalent method for forcibly holding tissue closed is using sutures or stitches, or staples.

A surgical suture or staple is a medical device used to ligate (tie-off) vessels, arteries, and fluid-carrying ducts by encircling the vessels and occluding flow within the vessel by tightening the suture around the vessel. Sutures or staples may also be placed to suspend or stabilize anatomic structures or to secure or locate implants in body tissue. The suture generally involves a needle with an attached length of thread. The sutures typically have two loose ends knotted together near the wounded tissue. The sutures are classified as absorbable and non-absorbable, where the absorbable sutures are gradually ingested or absorbed by the tissues. The non-absorbable sutures have to be removed manually after the wound is healed using assistors like scissors and the like.

The incorporation of sharp instruments such as scissors during the removal of the suture may sometimes lead to injuries to the tissue of the patient and the health care team. This is due to the misplacement, difficulty in using surgical supporters or assistors, and complexity of the access to a surgical region of the sharp instruments. Access may be impeded by limited space available to safely disconnect it from the body tissue. Approximately 15-33 percent of occupational injuries result from sharp exposure. About 4.4 percent of all new HIV cases today result from sharp exposure, of which 25 percent are nurses who reported injuries.

In current modern surgical procedures, sutures are used in many locations throughout the body, including internal sites accessed by endoscopic, laparoscopic, or orthoscopic tools. Depending on the location, access to the surgical site may be limited, creating difficulties with proper knot tying or crowding from other equipment in the surgical region of interest. These difficulties can lead to ineffective suture closure, multiple attempts at re-suturing, difficulties with the removal of the sutures, as well as increased closure times. Ultimately, these difficulties lead to increased surgical risks to the patient, occupational risks to the health care team as well as adverse economic effects derived from longer closure times.

Conventionally, a suture block may be used to secure surgical sutures without knots. See, e.g., U.S. Pat. No. 5,919,208, which is incorporated by reference. The device comprises a body in a discoidal shape which includes a female cavity and a male peg which are further coupled by snapping through force-fastening means. The male peg couples by forced insertion into the female cavity. The discoidal body comprises two semi-discoidal parts which can be mutually connected at an end by a hinge element. This device helps in holding sutures without damaging or mortifying the latter and reducing the pain caused by the knotted thread that may damage and constrict the tissue. The block offers the possibility of performing a suture without having to put down the instruments to free hands to tie the knots. But the hinge element that connects semi-discoidal parts limits suture or securement size or the number of sutures given in a fixed fulcrum.

U.S. Pat. No. 7,033,379 discloses a suture lock device with first and second engaging elements. They create a non-through bore capture zone that frictionally secures a suture. That patent is incorporated by reference. A male-engaging element includes a cylindrical projection, and the female-engaging element includes a cylindrical cavity. The cylindrical cavity is designed for physical coupling with a cylindrical projection. The frictional holding force secures the suture along the length of the suture in the capture zone between the mating elements. However, the sutures are removed manually after wound healing with sharp instrumental objects. This may lead to the pinching or cutting of skin or tissue and causes trauma to the tissue.

Therefore, there is a need for suture knotting devices that secures sutures to eliminate the need for manual knots. Such devices must be able to deploy, adjust and remove sutures without requiring any sharp objects or a suture removal kit. There is a need for a suture knot device that allows ease of use with limited dexterity with a minimal training period and can be deployed by the health care team with a favorable safety and efficacy profile. A suture knot device is needed which is easily adaptable and operable in instrument or organ crowded areas with varied thickness, stiffness, and material.

### SUMMARY OF THE INVENTION

Against this background, one objective of the disclosure is to provide a flat or rounded or ellipsoidal, or smooth device form with no sharp edges for a suture knotting capsule. The device reliably secures sutures in such a way as to eliminate the need for tying manual knots. As used herein the term “suture” embraces sutures, wires, tubes, and lines.

Another objective is to provide a suture knotting capsule that comprises cooperating male and female separable parts to secure a suture to a catheter or any device or tissue to the skin or any other body tissue.

A further objective is to provide separable parts which can be gently pulled or twisted apart to adjust or remove the suture, depending on the clinical indication.

Yet another objective is to provide a capsule knotting device that helps deploy and remove sutures without requiring sharp objects or a suture removal kit.

A still further objective is to provide a suture knotting device that allows a wide range of dexterity and maneuvering for healthcare team members with minimal training, ease, and less pressure. The device also allows the suture to be adjusted in real-time without the need for additional suture bite in the tissue/background

Another objective is to avoid pinching or cutting skin or trauma to tissue while removing a knot.

A further objective is to provide a suture knotting device that is easily adaptable and operable in instrument- or organ-crowded areas with varied thickness, stiffness, and material.

The disclosure proposes a device to cinch mid or distal-end regions of sutures with a reversible deployment and a quick-release mechanism. Following is a simplified summary to provide a basic understanding of some aspects of the claimed subject matter. This summary is not an extensive overview. It is not intended to identify key/critical elements or to delineate the scope of the claimed subject matter. Its sole purpose is to present some concepts in a simplified form as a prelude to the more detailed description presented later.

To overcome the above deficiencies of the prior art, disclosed is a device that is flat or rounded or ellipsoidal or cylindrical or is of a smooth form with no sharp edges. The device is adapted to secure tissue or any other surgical implement to the skin or body tissue. It eliminates the need for manual knots.

According to one embodiment, the cinch suture knotting device cinches suture ties with a reversible deployment and quick-release mechanism. The cinch suture knotting device comprises a male half, a female half, and a longitudinal projection with or without a plurality of ridges. The cinch suture knotting device allows reversible deployment and removal of the suture thread without requiring sharp objects. The cinch suture knotting device is slid close to the intended contact surface. The female half is fastened snugly onto the male half to trap the suture threads and thereby secure the system. The male half or the female half can be gently pulled or twisted away, either to release the suture threads or to adjust the suture thread. Preferably, the suture knotting device is coated with antimicrobial material in non-invasive placements and may or may not have bio-absorbable material for internal invasive placements.

The male half has a closed end. The female half is configured to allow both ends of the suture thread to pass with a blind distal end. Specifically, the male half and the female half each comprise a central lumen or canal which runs along the length of the male half and the female half to receive the suture threads. The suture threads can be passed from the central connecting ends of the female half to exit at either side of the halves.

The longitudinal projection extends from the end of the male half and has a tapered end to allow the female half to fit onto the male half easily and snugly. The tapered end of the longitudinal projection has a broad end at the base and a narrow end at the tip. The female half may comprise a wider central lumen or canal. The wider central lumen or canal houses the tapered end of the male half and thereby snugly fits at its base.

The male half and the female half can also be engaged either using a snapping mechanism or twisted mechanism or the like. The plurality of ridges or grooves or trenches or projections formed on the exterior and/or interior surface of the male half and the exterior and/or interior surface of the female half provide a gripping surface to a medical user. However, this design of the ridges could be reversed. Preferably, the device is made from a pliable material to accommodate stretch and conform to various projections or surfaces. In addition, the external surface of this device is smooth and atraumatic.

Further objects and advantages of the present invention will be apparent from a study of the following portion of the specification, the claims, and the attached drawings.

### BRIEF DESCRIPTION OF THE DRAWINGS

The accompanying drawings, which are incorporated in and constitute a part of the specification, illustrate an embodiment of the invention, and, together with the description, serve to explain the principles of the disclosure.

FIG. 1 is an exploded view of a suture knotting capsule or device in accordance with an exemplary embodiment.

FIG. 2 illustrates a front view of a cylindrical suture knotting capsule in accordance with an exemplary embodiment.

FIG. 3A illustrates the top views of the male and female halves of the capsule in accordance with an embodiment.

FIG. 3B illustrates the bottom views of the male and female halves of the capsule in accordance with an embodiment.

FIG. 4 illustrates an exploded view of a flat suture knotting capsule in accordance with an alternative embodiment.

FIG. 5A illustrates the top views of the male and female halves of a flat suture knotting capsule in accordance with the alternative embodiment.

FIG. 5B illustrates a bottom view of the male and female halves of a flat suture knotting capsule in accordance with the alternative embodiment.

FIGS. 6-18 are a stepwise series of drawings illustrating a method of using the suture knotting capsule to secure end regions of a suture.

### DETAILED DESCRIPTION

Various embodiments will be described in reference to the accompanying drawings. Wherever possible, the same or similar reference numerals are used in the drawings and the description to refer to the same or like parts or steps.

The present disclosure solves problems arising from the prior art described above. It is an object of the present disclosure to provide a flat or rounded or ellipsoidal or cylindrical, or smooth form of device with no sharp edges to secure a catheter or any other device to the skin or any body tissue and to thereby eliminate manual knots and need for sharp instruments for removal.

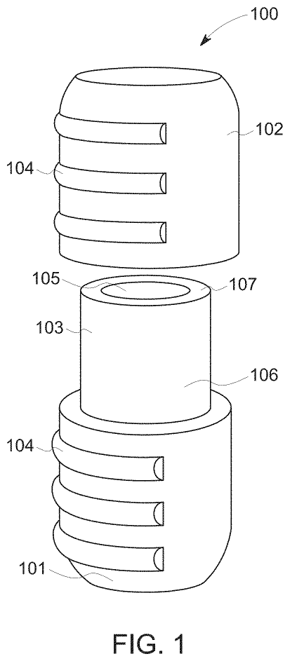

According to an exemplary embodiment, FIG. 1 refers to a suture knotting capsule or device 100. The suture knotting capsule 100 comprises a male half 101, a female half 102, a longitudinal projection 103, and a plurality of ridges 104. It will be appreciated that the term ‘half’ is used in a non-limiting manner.

The suture knotting device 100 allows reversible adjustment, deployment, and removal of the suture thread without requiring any sharp objects.

The male half 101 has a closed end. The female half 102 is configured to allow both ends of the suture thread to pass through. It has a blinded distal end. Specifically, the male half 101 and the female half 102 each comprise a central lumen 105 or canal which runs along the length of the male half 101 or the female half 102 to receive the suture threads. Both ends of the suture threads can pass from the central connecting ends of the female half 102 to exit at either side of the halves.

The longitudinal projection 103 is preferably formed or extruded at the end of the male half 101. It has a tapered end that allows the female half 102 to fit onto the male half easily and snugly 101. Specifically, the tapered end of the longitudinal projection 103 has a broad end at the base 106 and a narrower end at the tip 107. This allows the female half 102 to easily cooperate with the male half 101 to form one unit that is variously referred to herein as a suture knot capsule or device.

The female half 102 comprises a wider central lumen or canal 105. The wider central lumen or canal 105 houses the tapered end of the male half 101 and thereby snugly fits at its base. The male half 101 and the female half 102 can also cooperate by using a snapping mechanism or twisted mechanism or the like. The plurality of ridges 104 formed on the exterior surface of the male half 101 and the female half 102 provide a gripping surface to the medical user. The plurality of ridges on the interior surface would permit sutures/lines/tubes/wires of different caliber to get snugly trapped.

After receiving the suture ends, the suture knotting device 100 may be slid closer to an intended contact surface. The female half 102 is fastened snugly onto the male half 101 to trap the suture threads and thereby secure the system. The male half 101 or the female half 102 can be gently pulled or twisted away, either to release the suture threads or to adjust a knot. Preferably, the suture knotting device 100 can be coated with antimicrobial material in non-invasive placements and have (optionally) bio-absorbable material in invasive placements.

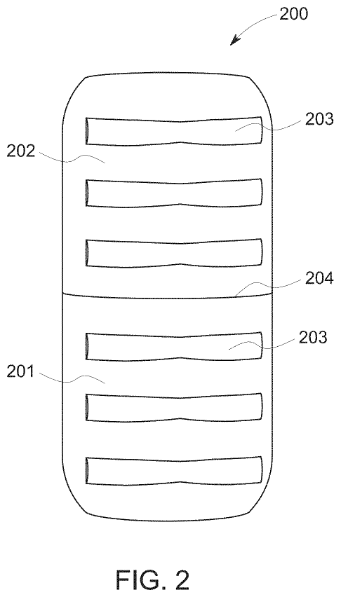

FIG. 2 depicts a front view of the suture knotting capsule 200 where the male half 201 is snugly fitted to the female half 202 at the connecting end 204. The outer surface of the suture knotting capsule has a plurality of ridges 203 to provide a gripping surface to the medical user.

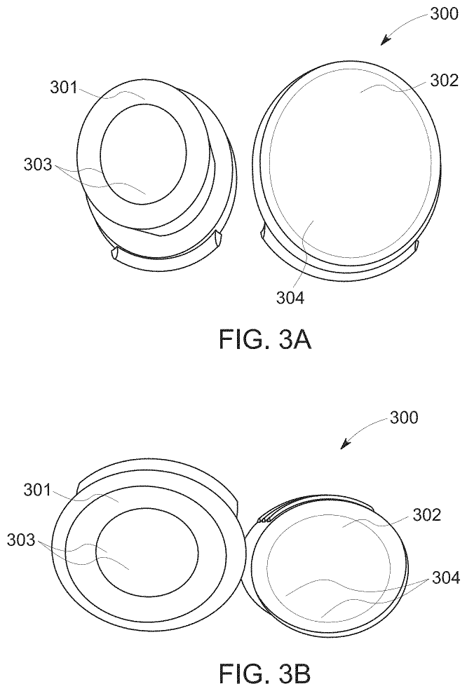

According to another exemplary embodiment of the invention, FIGS. 3A and 3B depict top and bottom views of the male and female halves of the suture knotting capsule 300. The female half 302 is a cap-like structure that comprises a wider lumen or canal with an open end 304 that optionally provides a slot for suture threads and houses gradually tapering male projection where the female half 302 snugly fits on the male half 301 at its base.

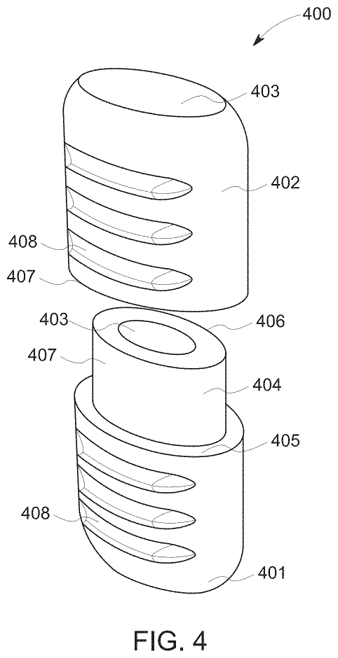

According to another exemplary embodiment, FIG. 4 is an exploded view of a flat suture knotting capsule 400. The male half 401 has a closed end. The female half 402 is configured to allow each end of the suture threads to pass therethrough. Specifically, the female half 402 comprises an open end 403 with central lumen or canal that accommodates the suture thread. The longitudinal projection 404 at the end of the male half 401 has a tapered end that allows the female half 402 to easily snugly fit onto the male half 401. The tapered end of the longitudinal projection 404 has a broad end at the base 405 and a narrow end at the tip 406 which allows the female half 402 to easily snug fit with the male half 401 to form one unit i.e., a capsule knot. The plurality of ridges 408 are formed on the exterior surface of the male half 401 and the female half 402 provide a gripping surface to the medical user.

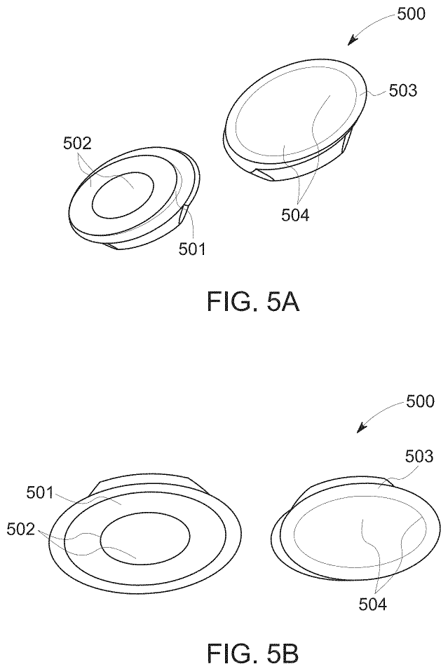

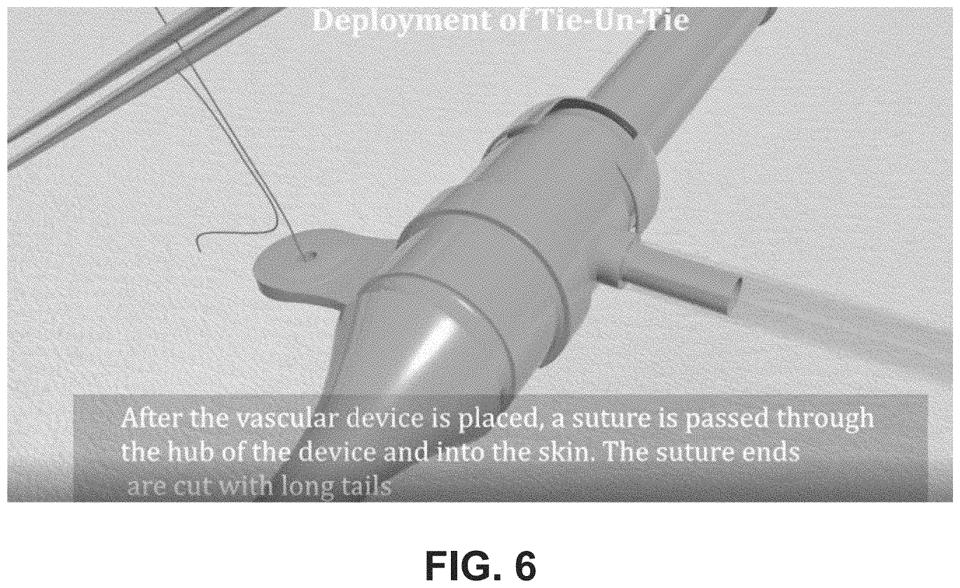

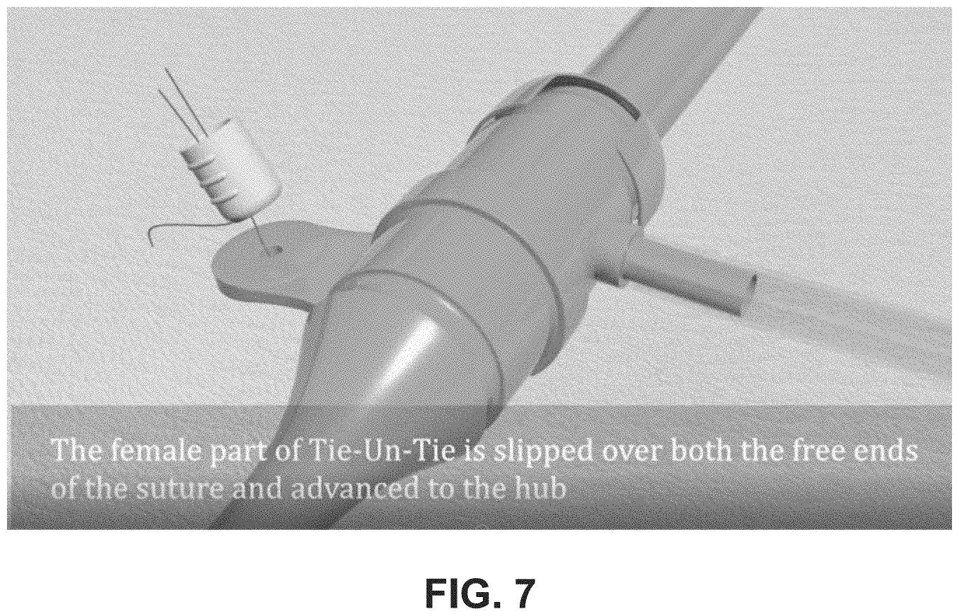

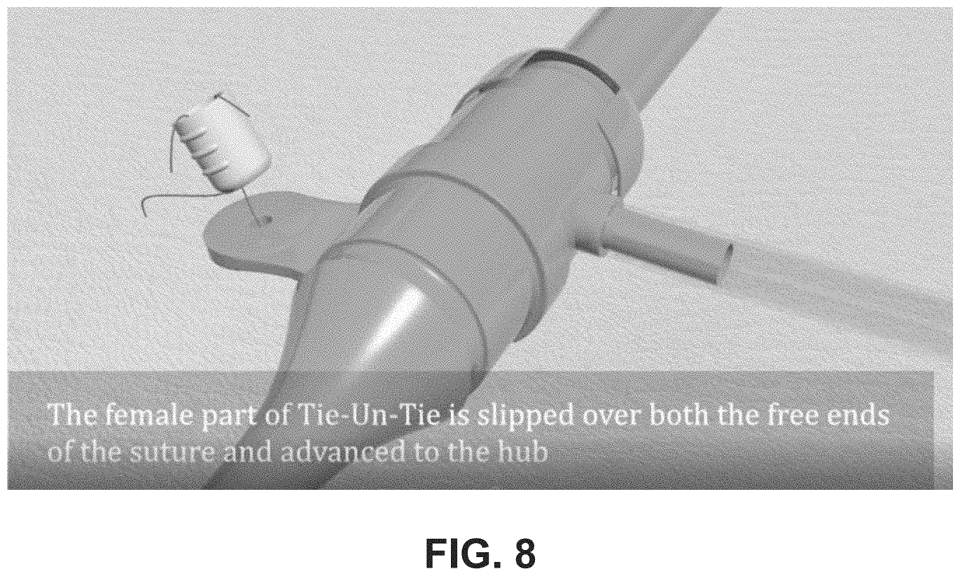

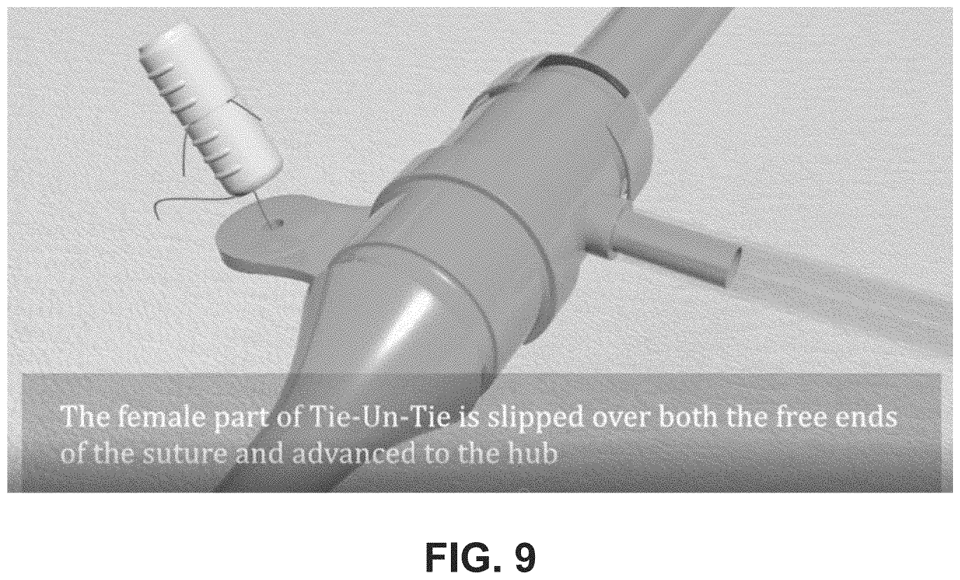

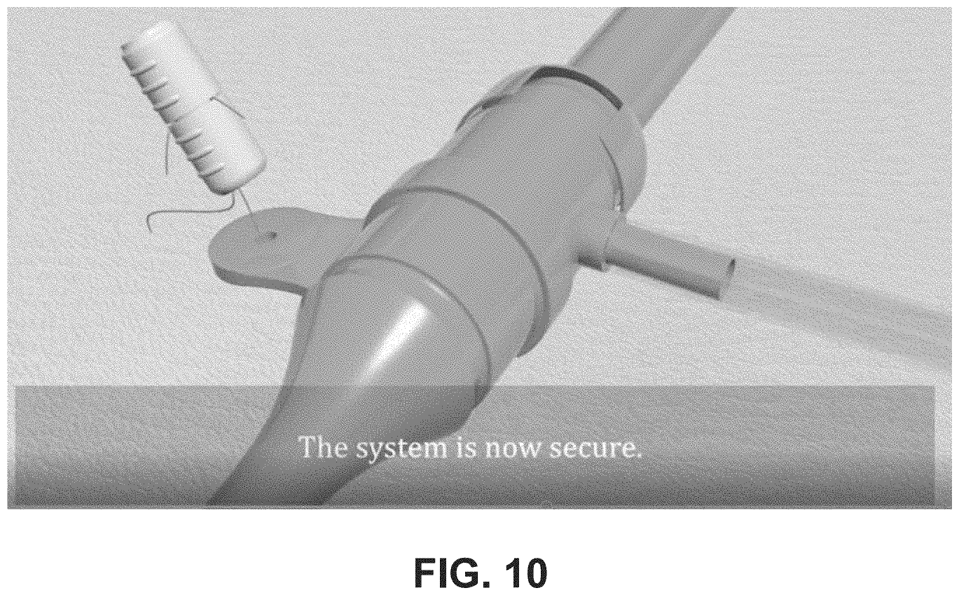

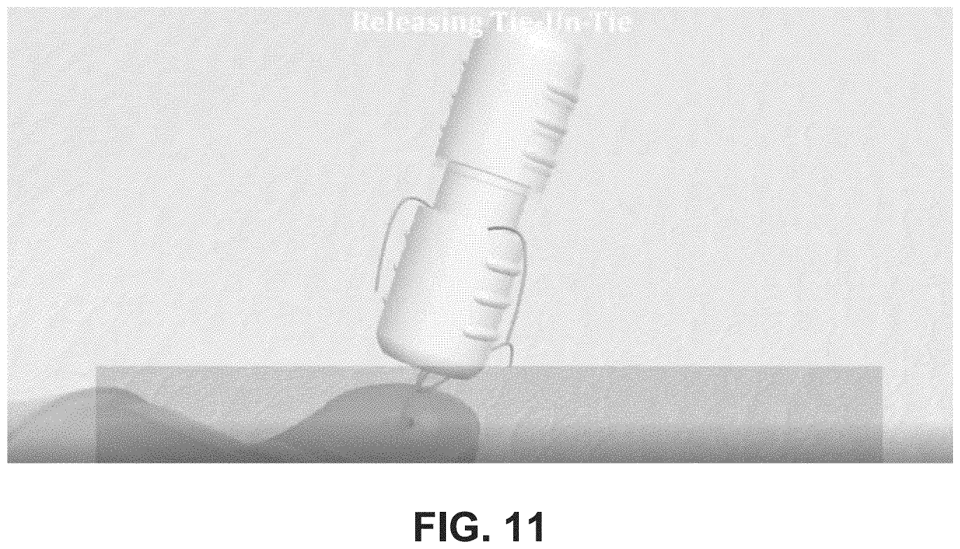

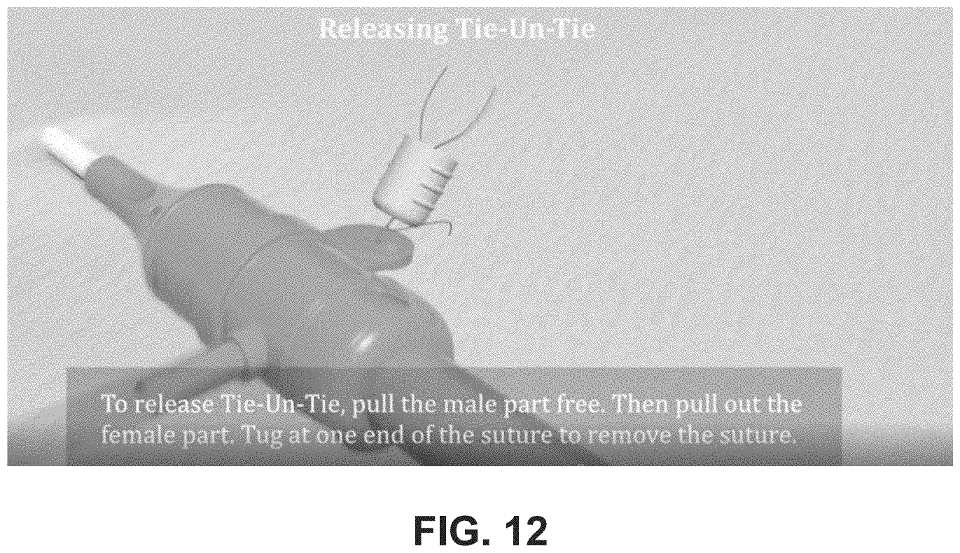

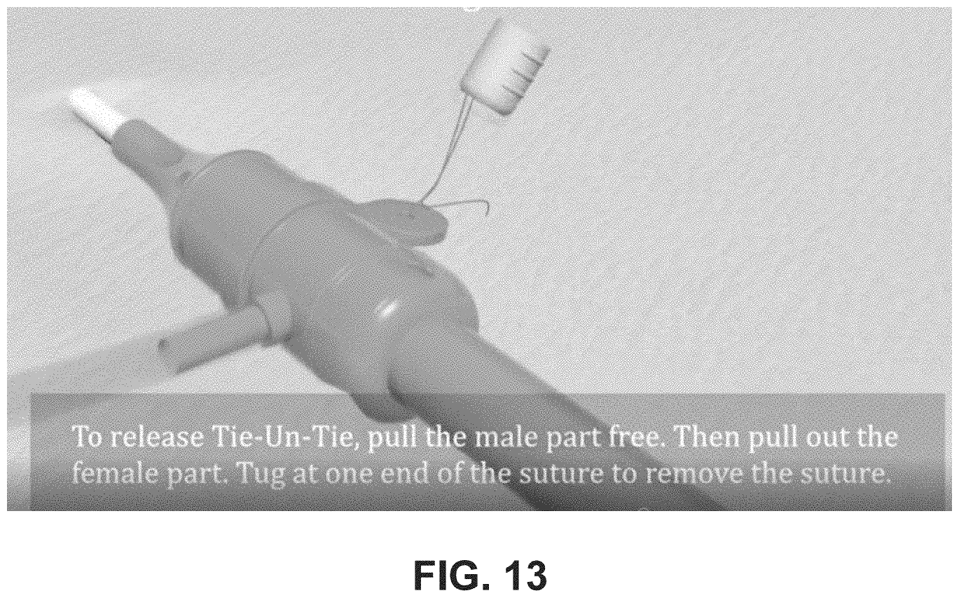

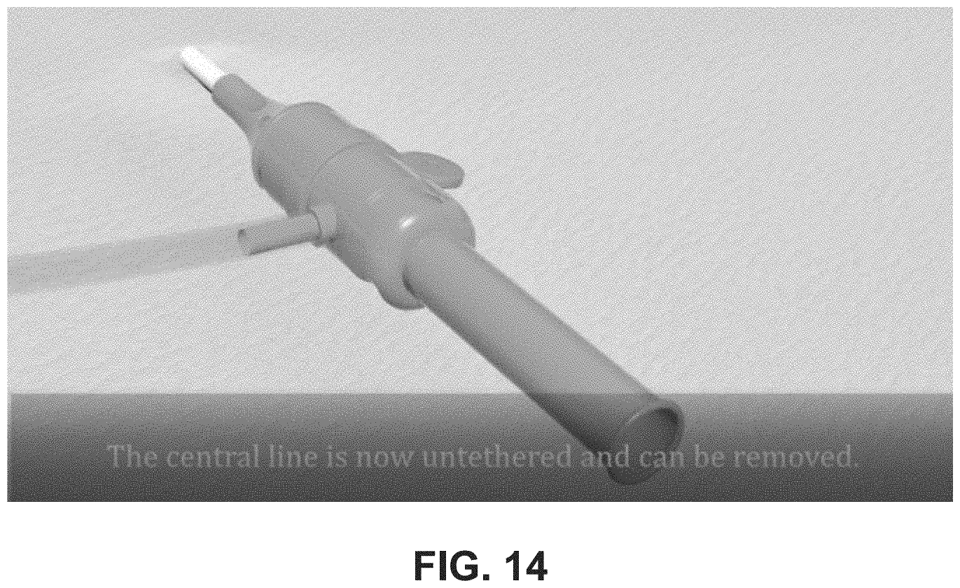

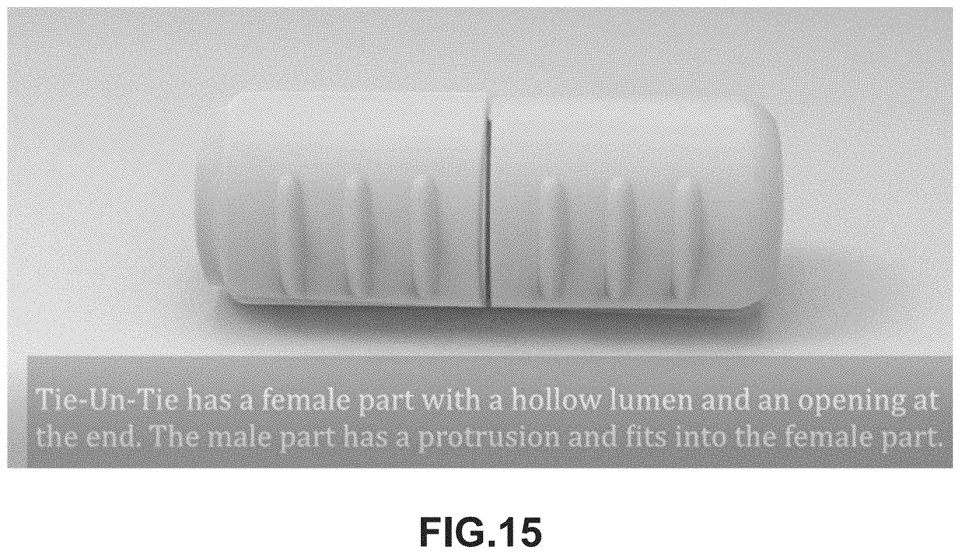

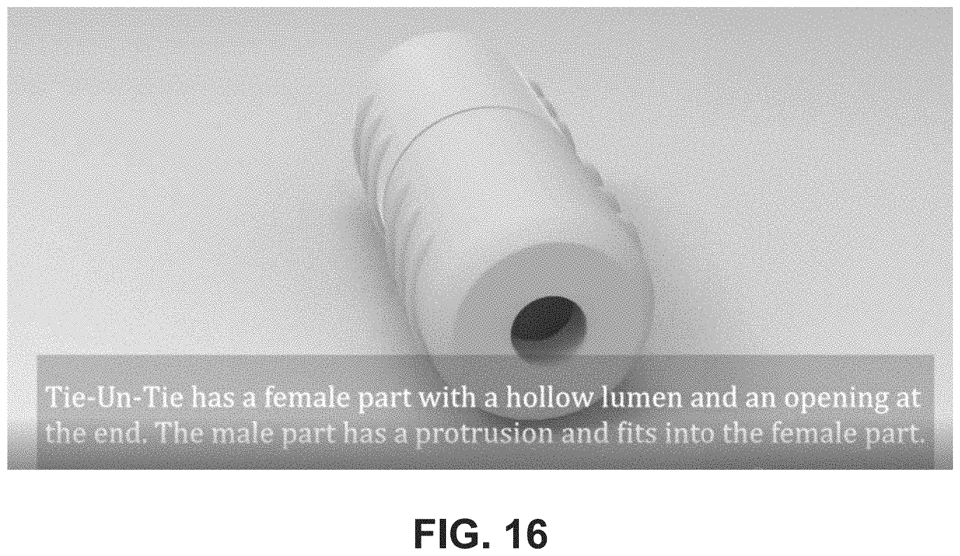

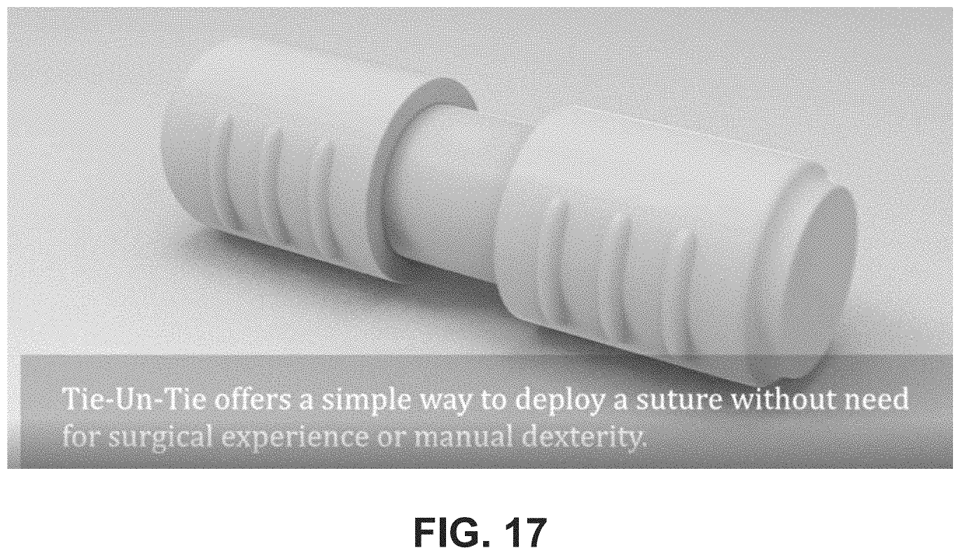

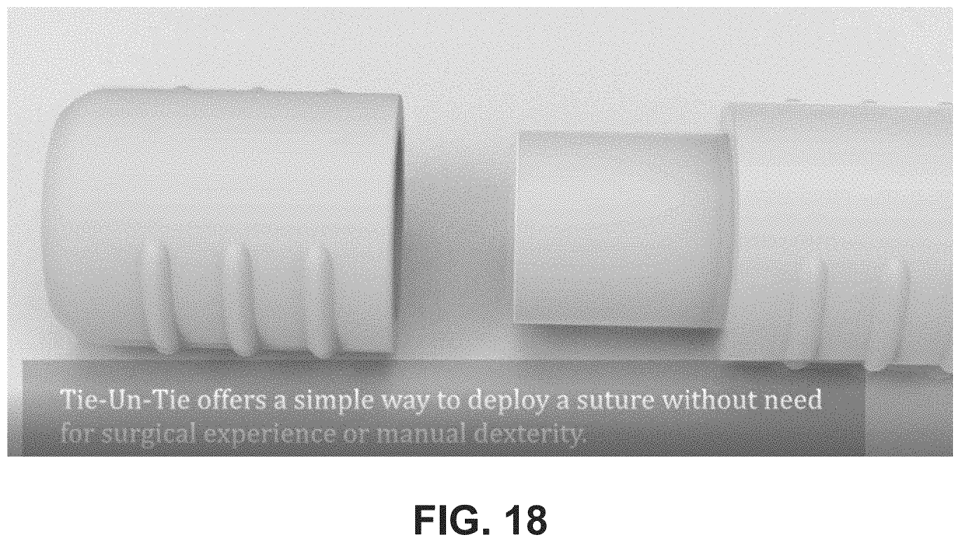

FIGS. 5A and 5B refer to the top and bottom views of the male and female halves of the suture knotting capsule 500. The male half 501 has a lumen or canal with a closed end 502. Further, the female half 503 is a cap-like structure with a wider lumen or canal functioning with an open end 504 that acts as a slot for suture threads. The female half 503 accommodates a gradually tapering male projection so that the female half 503 snugly fits on the male half 501 at its base. Specifically, the male 501 and female halves 503 assume a relatively flat form factor.

Numerous advantages of the present disclosure may be apparent from the discussion above. In accordance with the present disclosure, a flat or cylindrical or circular, or universally shaped suture knotting capsule secures sutures, permits real-time adjustment, and eliminates the need for manual knots. The suture knotting capsule further comprises male and female separable parts to secure a catheter or any device to the skin or any body tissue. Such separable parts can gently be pulled or twisted apart to adjust or remove the suture depending on the clinical condition to thereby avoid pinching or cutting of skin or trauma to tissue while removing the suture thread. The suture knotting capsule helps deploy, adjust and remove sutures without requiring sharp objects or a suture removal kit. It allows a wide range of dexterity and maneuvering for healthcare team members with ease and less pressure. The suture knotting capsule is easily adaptable and operable in instrument or organ crowded areas with varied thickness, stiffness, and material.

It will readily be apparent that numerous modifications and alterations can be made without departing from the principles underlying the invention, and all such modifications and alterations are intended to be embraced by this application.

What is claimed is:

1. A cinch suture knotting device, comprising:
a male half having a closed end;
a female half configured to cooperate with both ends of a suture thread, the female half having a blind distal end;
a longitudinal projection extending from an end of the male half, the longitudinal projection having a tapered end that allows the female half to snugly fit onto the male half; and
a plurality of ridges formed on an exterior surface of the male half and an exterior surface of the female half that provide a gripping surface to a medical user,
wherein the suture knotting device allows reversible deployment and removal of the suture thread without requiring any sharp objects.

2. The cinch suture knotting device as recited in claim 1, wherein the female half comprises a central lumen or canal which runs along the length of the female half to receive the suture threads.

3. The cinch suture knotting device as recited in claim 1, wherein the suture threads pass from a central connecting end of the female half.

4. The cinch suture knotting device as recited in claim 1, wherein the tapered end of the longitudinal projection has a broad end at a base of the projection and a narrow end at a tip.

5. The cinch suture knotting device as recited in claim 1, wherein the female half has a wider central lumen or canal adapted to receive the projection extending from the male half.

6. The cinch suture knotting device as recited in claim 5, wherein the wider central lumen or canal houses the tapered end of the male half.

7. The cinch suture knotting device as recited in claim 4, wherein the male half and the female half are adapted to house a snap-on mechanism or twisted mechanism or the like.

8. The cinch suture knotting device as recited in claim 1, wherein the suture knotting capsule is slid closer to an intended contact surface and the female half is fastened snugly onto the male half to trap the suture threads.

9. The cinch suture knotting device as recited in claim 1, wherein either the male half or the female half can be gently pulled or twisted away either to release or adjust the suture threads.

10. The cinch suture knotting device as recited in claim 1, wherein the suture knotting capsule is coated with antimicrobial and/or bio-absorbable material.

11. A cinch suture knotting device, comprising:
a male half having a closed end;
a female half configured to allow both ends of a suture thread to pass, the female half having a blind distal end;
a longitudinal projection extending from the end of the male half, the longitudinal projection having a tapered end that allows the female half to snugly fit onto the male half; and
a plurality of ridges formed on an exterior surface of the male half and an interior and exterior surface of the female half that provide a gripping surface to a medical user,
wherein the suture knotting device allows reversible deployment and removal of the suture thread without requiring any sharp objects.

12. A method for using a cinch suture knotting device comprising the steps of
threading opposing ends of a suture through a female half of the cinch suture knotting device;
positioning the female half of the device in relation to an anatomical site so as to absorb a desired amount of slack in the opposing ends;
snugly fitting a male half of the device onto the female half over the opposing ends, thereby frictionally engaging the opposing ends.

13. The method of claim 12, further comprising the steps of:
separating the male from the female halves, thereby loosing and releasing frictional forces that secured the opposing ends so that a quick release feature is enabled.
